(19) United States
(12) Patent Application Publication
(10) Pub. No.: US 2024/0260795 A1
(43) Pub. Date: Aug. 8, 2024

(54) SELF-CLEANING HAND DRYER

(71) Applicant: Dyson Technology Limited, Wiltshire (GB)

(72) Inventors: Gemma MCLUCKIE, Poole (GB); Dominic Jan ZOLKIEWKA, Swindon (GB)

(21) Appl. No.: 18/294,305

(22) PCT Filed: Jun. 28, 2022
(86) PCT No.: PCT/GB2022/051655

§ 371 Date: Feb. 1, 2024

(30) Foreign Application Priority Data
Aug. 3, 2021 (GB) 2111182.8

(51) Int. Cl. A47K 10/48; A61L 2/10; A61L 2/24

(52) U.S. Cl. CPC A47K 10/48; A61L 2/10; A61L 2/24; A61L 2202/11; A61L 2202/14

(57) ABSTRACT

A hand dryer is provided comprising at least one part that is susceptible to contamination when touched, and at least one light source for emitting light in a far UVC portion of the electromagnetic spectrum, the at least one light source being arranged in such a way as to illuminate the at least one part for the decontamination thereof, when emitting the light.

### TECHNICAL FIELD

The present invention relates to a hand dryer.

### BACKGROUND

In public bathrooms and other places where many different people wash their hands, it is very common to have some kind of hand drying device installed for people to dry their wet hands. An important design concern for such hand dryer devices is to minimise the amount of bacteria and other microbes it may pass on from one user to another. For example, towel dispensers are designed such that the user only has to touch the towel to dry his hands. Often, the towel can be thrown away after use or automatically rolls up into an interior of the towel dispenser. To avoid the use of towels, many different types of hand dryers using air blowers are on the market. While older models tend to have a large button for activating the blower, newer air blowers are typically activated using proximity sensors for detecting the presence of a user's hands.

However, even then, contamination still occurs and regular cleaning of the hand dryer is needed to avoid health risks for its users. Unfortunately, the cleaning of such hand dryers often involves the use of a wet cloth that has already been used for cleaning other surfaces too. Often such cleaning methods may be successful in removing visible dirt, but may add more or different microbes to a surface than it removes.

As a consequence, there is a need for ways to better protect the users of hand dryers against contact with unwanted microbes while drying their hands.

### SUMMARY OF THE INVENTION

According to an aspect of the invention there is provided a hand dryer comprising at least one part that is susceptible to contamination when touched, and at least one light source for emitting light in a far UVC portion of the electromagnetic spectrum, the at least one light source being arranged in such a way as to illuminate the at least one part for the decontamination thereof, when emitting the light.

The ultraviolet portion of the visual spectrum is typically defined as spanning the range of about 180 to 400 nm. In particular, the UVC, or far UVC range spans from 180-280 nm Light of these wavelengths is known to be very effective in killing any microbes that may have accumulated on the illuminated surfaces. Although such light is known to be used in light fixtures used for cleaning rooms in hospitals and in standalone curing lights used for 3D printer resin and nail polish, it has so far not been used in hand dryers or for cleaning contaminated parts of hand dryers. For example, 222 nm light does not damage the material of the surfaces it illuminates. This is especially advantageous because most floor care devices are at least partially made of plastics that are easily damaged by other particular wavelengths of UV light. In addition thereto, the wavelength is not harmful to humans. Another important advantage of the 222 nm light is that no direct line of sight between the light source and the surface or part to be cleaned is needed. Indirect irradiation of the 222 nm light helps to get rid of the microbial contamination too. In addition thereto, the light source may be coupled with a visible light which makes it clear to the user and other bystanders when the device is operating in a cleaning mode.

It is to be noted that emitting light in a far UVC portion of the visual spectrum as part of a decontamination process means that the emitted light contains a significant portion of light in that part of the electromagnetic spectrum and that the intensity of that significant portion is sufficient to have a useful anti-microbial and decontaminating effect. The emitted light does not need to be exclusively in the far UVC portion of the electromagnetic spectrum. As long as there is a sufficient intensity of light in that portion of the spectrum, and preferably at or around the 222 nm wavelength, for achieving a decontaminating effect, light of other parts of the electromagnetic spectrum may be emitted too. Further it is noted that, as part of the decontamination process, the intensity of the emitted light may vary over time. Such variations may be gradual and continuous or in the form of a pattern of light pulses. If pulsed light is used, the frequency, duration and intensity of the pulses may either be constant or varying.

The emitted light is preferably coupled with a source for at least illuminating those parts of the hand dryer that tend to attract a lot of microbial contamination or that have a larger probability of passing on such contamination to its users. Therefore, in a preferred embodiment, the at least one part comprises a nozzle for blowing out a hand drying air stream. During use, these nozzles may receive microbial contamination coming from the user's hands that may come very close to or even in direct contact with the nozzles. Conversely, microbial contamination located at or near the nozzles can easily be blown onto the next user's hands. When the hand dryer is cleaned with a wet cloth, it is highly likely that at least the nozzles are wiped. By cleaning the nozzles with far UVC light, the spread of microbial contamination via the nozzles can be significantly reduced.

Preferably, the hand dryer further comprises a controller, operatively coupled to and configured for timed control of the at least one light source. For example, the light source may be activated for a set amount of time, after each use of the hand dryer. Alternatively, the light source may be activated whenever the room lighting is switched off and no one is in the room. The light source may then keep active for a set amount of time, or until the room lighting is switched on again.

Another option is to start a cleaning cycle directly after the hand dryer has been cleaned by hand. For this purpose, the hand dryer may comprise a decontamination button, operatively coupled to the controller, the controller being configured to activate the at least one light source in response to activation of the decontamination button.

Most microbial contamination will occur when the at least one part is touched, which may happen either during use or during cleaning. Therefore, the hand dryer may further comprise at least one touch sensor for detecting a touch of the at least one part, the touch sensor being operatively coupled to the controller, the controller being configured to activate the at least one light source in response to detecting the touch of the at least one part. Such touch sensors may, e.g., be mechanical sensors or electronic sensors, such as capacitive or resistive touch sensors. Optionally, the touch sensor is configured to detect a touch of a portion of the at least one part, the controller being configured to activate the at least one light source to selectively illuminate the touched portion. This will make it possible to only illuminate those portions of the at least one part that are actually touched and have the highest risk of being contaminated, thereby improving the energy efficiency of the cleaning operation.

Additionally, the hand dryer may comprise a contamination detector, operatively coupled to the controller, for detecting contamination on a contaminated portion of the at least one part, the controller being configured to selectively illuminate the contaminated portion. The contamination detector may use an optical sensor measuring the reflectance of light of a particular wavelength at a surface of the at least one part. The light used for this reflection measurement may be the light from the at least one light source, but may also be provided by a separate light source, possibly emitting light at a different wavelength.

In preferred embodiments, the hand dryer comprises a fan, operatively coupled to the controller, for generating a hand drying air stream. While the light source is emitting light for cleaning the at least one part, the controller may temporarily disable the fan to avoid the hand dryer being used and recontaminated before the cleaning process is completed. However, in other embodiments or modes of operation, the hand dryer may be used while the at least one light source emits its far UVC light.

### BRIEF DESCRIPTION OF THE DRAWINGS

Embodiments of the invention will now be described by way of example with reference to the accompanying drawings, in which:

FIG. 1 shows an exemplary embodiment of a hand dryer according to the invention.

FIG. 2 schematically shows a cross section of the hand dryer of FIG. 1.

FIG. 3 shows a further exemplary embodiment of a hair dryer according to the invention.

### DETAILED DESCRIPTION

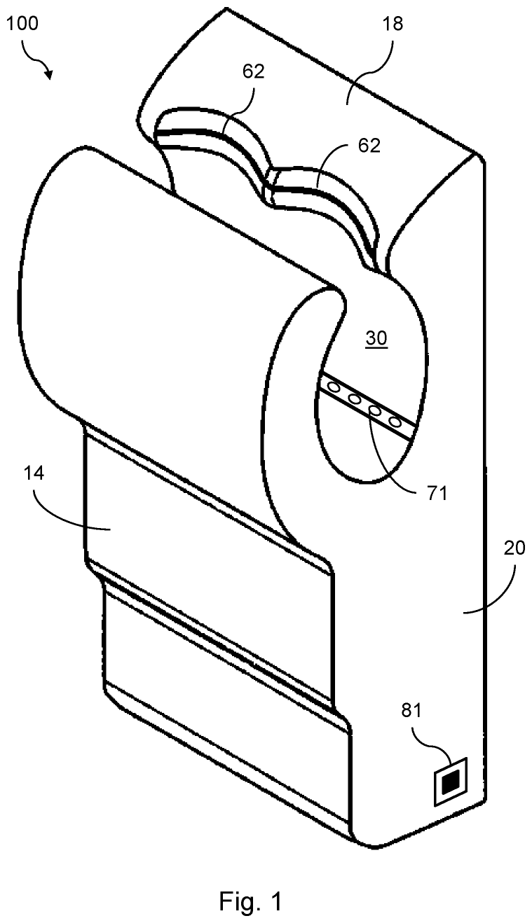

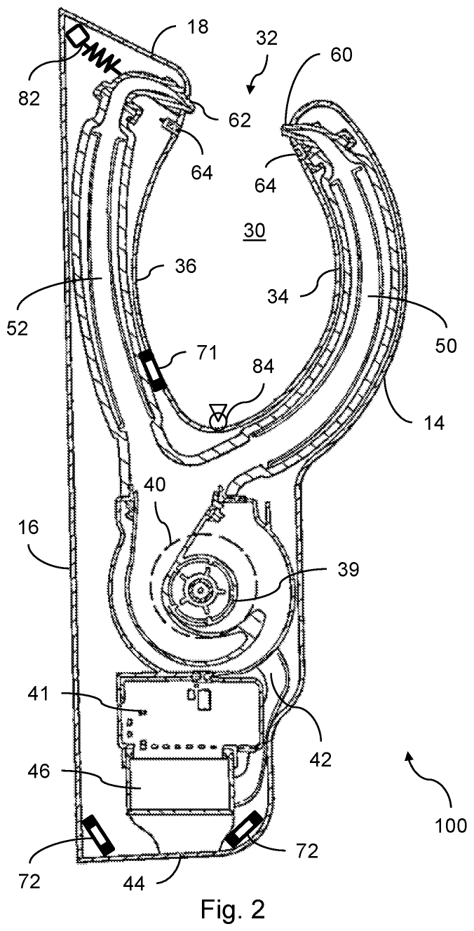

FIGS. 1 and 2 show an exemplary embodiment of a hand dryer 100 according to the invention. The hand dryer 100 comprises an outer casing having a front wall 14, a rear wall 16, an upper face 18 and side walls 20. The rear wall 16 can incorporate fixing devices for securing the hand dryer 100 to a wall or other structure prior to use. An electrical connection may be provided on the rear wall or elsewhere on the casing. A cavity 30 is formed in the upper part of the casing. The cavity 30 is open at its upper end and delimited thereat by the top of the front wall 14 and the front of the upper face 18. The space between the top of the front wall 14 and the front of the upper face 18 forms a cavity entrance 32 which is sufficiently wide to allow a user's hands to be introduced to the cavity 30 through the cavity entrance 32. The cavity 30 is also open to the sides of the hand dryer 100 by appropriate shaping of the side walls 20. The cavity 30 has a front wall 34 and a rear wall 36 which delimit the cavity 30 to the front and rear respectively.

As shown in FIG. 2, a motor 39 is located inside the casing and a fan 40, which is driven by the motor 39, is also located inside the casing. The motor 39 is connected to the electrical connection and controlled by a controller 41. The inlet 42 of the fan 40 communicates with an air inlet 44 formed in the casing. A filter 46 is located in the air passageway connecting the air inlet 44 to the fan inlet 42 so as to prevent the ingress of any debris which might cause damage to the motor or the fan 40. The outlet of the fan 40 communicates with a pair of air ducts 50, 52 which are located inside the casing. The front air duct 50 is located primarily between the front wall 14 of the casing and the front wall 34 of the cavity 30, and the rear air duct 52 is located primarily between the rear wall 16 of the casing and the rear wall 36 of the cavity 30.

The air ducts 50, 52 are arranged to conduct air from the fan 40 to a pair of opposed slot-like openings or nozzles 60, 62 which are located in the front and rear walls 34, 36 respectively of the cavity 30. The nozzles 60, 62 are arranged at the upper end of the cavity 30 in the vicinity of the cavity entrance 32. The slot-like openings 60, 62 are each configured so as to direct an airflow generally across the cavity entrance 32 towards the opposite wall of the cavity 30. The slot-like openings 60, 62 are offset in the vertical direction and angled towards the lowermost end of the cavity 30.

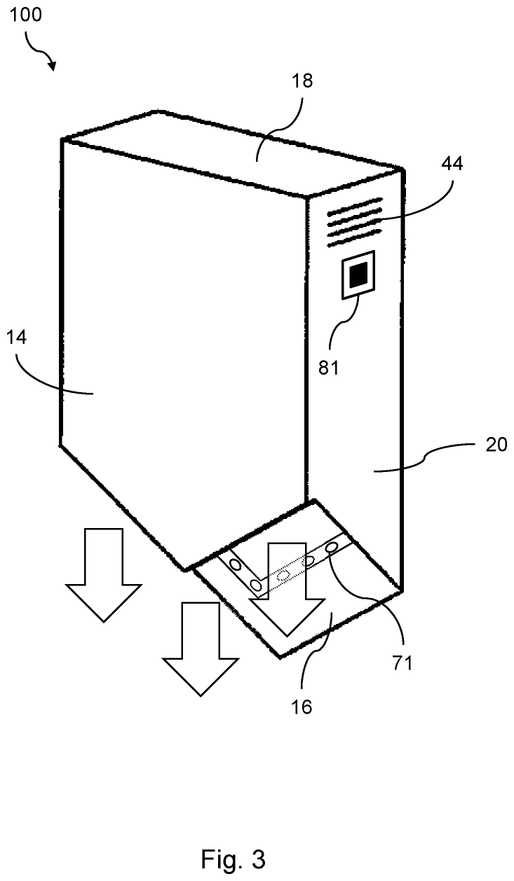

Proximity sensors 64 are positioned in the front and rear walls 34, 36 of the cavity 30 immediately below the slot-like openings 60, 62. These proximity sensors 64 detect the presence of a user's hands which are inserted into the cavity 30 via the cavity entrance 32 and are arranged to send a signal to the motor when a user's hands are introduced to the cavity 30. As can be seen from FIGS. 1 and 3, the walls 54a, 54b, 56a, 56b of the ducts 50, 52 project slightly beyond the surface of the front and rear walls 34, 36 of the cavity 30. The inward projection of the walls 54a, 54b, 56a, 56b of the ducts 50, 52 reduces the tendency of the user's hands to be sucked towards one or other of the walls 34, 36 of the cavity, which enhances the ease with which the hand dryer 10 can be used. The positioning of the proximity sensors 64 immediately below the inwardly projecting walls 54a, 54b, 56a, 56b of the ducts 50, 52 also reduces the risk of the proximity sensors 64 becoming dirty and inoperative.

When a user's hands are first inserted into the cavity 30 through the cavity entrance 32, proximity sensors 64 detect the presence of the user's hands and the controller 41 sends a signal to the motor 39 to drive the fan 40. The high speed rotation of the fan 40 causes air to be drawn into the hand dryer 10 via the air inlet 44. The air passes through the filter 46 and along the fan inlet 42 to the fan 40. The airflow leaving the fan 40 is divided into two separate airflows; one passing along the front air duct 50 to the slot-like opening 60 and the other passing along the rear air duct 52 to the slot-like opening 62.

According to an embodiment of the invention, the hand dryer 100 further comprises a light source 71, 72, preferably in the form of an array of LEDs (light emitting diodes), for emitting light in a far UVC portion of the electromagnetic spectrum. In this embodiment, an LED strip 71 is integrated in or attached to the rear wall 36 of the cavity 30. The light strip 71 is coupled to the controller 41, which controls the activation and deactivation of the light strip 71. For example, the light strip 71 may be activated for a set amount of time, after each use of the hand dryer 100, or after, e.g., every tenth time the hand dryer 100 is used. Alternatively, the light strip 71 may be activated whenever the room lighting is switched off and no one is in the room. The light strip 71 may then keep active for a set amount of time, or until the room lighting is switched on again. When turned on, this light strip 71 illuminates the cavity 30, the front and rear walls 34, 36 and the exposed portions of the proximity sensors 64 and the air nozzles 60, 62.

The ultraviolet portion of the visual spectrum is typically defined as spanning the range of about 180 to 400 nm. In particular, the UVC, or far UVC range spans from 180-280 nm. The light strip may thus use 222 nm LEDs as are readily available for other applications. One of the advantages of using light in the far UVC portion of the electromagnetic spectrum is that no direct line of sight between the light source and the surface or part to be cleaned is needed. Parts that are not directly illuminated by light from the light strip 71, but only by light reflected at the surfaces of the front and rear walls 64, 66 of the cavity 30, are thus effectively cleaned too. Preferably, the surfaces of the front and rear walls 64, 66 are reflective for light at the wavelength of the light emitted by the light strip 71 for ensuring optimal cleaning performance.

A second far UVC light source 72 is provided inside the hand dryer casing for enabling decontamination of the filter 46. When using the hand dryer 100, air is drawn in through the air inlet 44 and filtered by the filter 46 to avoid dust and dirt getting into the motor 39 or the fan 40. Over time, the filtered dust and dirt may accumulate at the filter surface. Now and then, the filter 46 may be removed for a thorough cleaning or replaced by a new one. In the meantime, however, microbes may accumulate and multiply at the filter surface 46 too. By illuminating the filter 46 with far UVC light, such microbes can be effectively eliminated to avoid them getting sucked into the airstream and being expelled through the nozzles 60, 62. Because the second light source 72 is located inside the casing, the light it emits may not be visible from the outside. The light source 72 may, e.g., be operated continuously or at regular intervals and for a set amount of time. Alternatively, it may be turned on for a predetermined period every time the hand dryer 100 is used, or after, e.g., every tenth time the hand dryer is used.

Additional light sources may be provided for illuminating, e.g., a drip tray of the hand dryer 100 or the immediate surroundings of the hand dryer 100. When used, water droplets from the user's hands may end up at the wall surface to which the hand dryer 100 is attached or on the floor surface below the hand dryer 100. When ensuring that such wall and floor surfaces are illuminated too, those surfaces can be cleared of bacterial and microbial contamination too.

As described above, the activation of the light sources 71, 72, is controlled by the controller 41 and may occur at regular intervals and for predetermined amounts of time. Alternatively, the activation of the light sources 71, 72 is triggered by the use of the hand dryer 100 or by the presence (or absence) of people in the room. Another option is to link activation of the light sources 71, 72 to the occurrence of a more traditional manual cleaning of the hand dryer 100. For example, a cleaner may, after having manually cleaned the hand dryer 100 use a button 81 to turn on the light strip 71 and/or the second light source 72. Such a button 81 is preferably located out of sight for normal users, using the hand dryer 100 to dry their hands, in order to avoid it being pressed by non-authorised people. To ensure that only authorised people can turn on the light sources 71, 72, e.g., a key or RFID tag may be used for their activation.

Most microbial contamination will occur when the hand dryer 100 is touched, which may happen either during use or during cleaning. Therefore, the hand dryer 100 may further comprise at least one touch sensor 82 for detecting a touch of a frequently touched part, such as the nozzles 60, 62. The touch sensor 82 is operatively coupled to the controller 41 for allowing, the controller 41 to activate the light strip 71 in response to detecting the touch of the nozzles 62, 60. In this example, the nozzle 62 mounted that it is slightly movable in a forward and backward direction. In normal use, a spring or other biasing element biases the nozzle in a more forward position. When touched, e.g., by a hand during use of the hand dryer 100 or by a wet cloth during cleaning, the nozzle is pushed backward, which is registered by the touch sensor 82 and communicated to the controller 41, which may then decide to start a cleaning program and turn on the light strip 71. Other types of touch sensors, such as electrical or optical touch sensors may also be used for this purpose.

Optionally, the touch sensor 82 is configured to detect a touch of only a portion of the nozzle 60, 62 and the controller activates only a corresponding portion of the light strip 71 in order to selectively illuminate the touched portion of the nozzle 60, 62. This may, e.g., be useful after a short and involuntary touch of the nozzle 60, 62 by a user who is drying his or her hands and may reduce the amount of emitted light and energy used for decontaminating the nozzle 60, 62.

Additionally, the hand dryer 100 may comprises a contamination detector, here in the form of a camera 84, operatively coupled to the controller 41, for detecting contamination on a contaminated portion of the parts that may be contaminated. This will allow the controller to selectively illuminate only the contaminated portion. Instead of a camera 84 a simpler optical sensor may be used, for example an optical sensor measuring the reflectance of light of a particular wavelength. The optical sensor may be integrated in the light strip 71 or in the proximity sensor 64, but can also be a separate unit. It may or may not use light of the same wavelength as emitted by the light strip 71.

By using low energy LED light in the far UVC portion of the electromagnetic spectrum, it is possible to eliminate the microbial contamination without damaging the material of the hand dryer, typically plastics or a light-weight metal such as aluminium. With this light, thorough decontamination of the illuminated parts may take at least 30 minutes to 2 hours and possibly more. The amount of time needed for an acceptable level of decontamination will depend on the intensity of the light in the relevant part of the visual spectrum. To avoid further contamination during the cleaning process, the fan may be disabled during the cleaning process. Because the light strip 71 and the second light source 72 deal with different contamination sources and are provided for cleaning different parts, they may not always be used at the same time and for the same duration.

In various modes of operation, the hand dryer 100 may also be used while the at least one light strip 71 and/or the second light source 72 are active. The light from the second light source 72 will typically not be visible from the outside unless, apertures or transparent materials are applied to the casing. Such apertures and transparent materials may be strategically designed for, e.g. branding and aesthetic purposes, in addition to allowing the user to easily identify when the second light source 72 is active.

FIG. 3 shows a further exemplary embodiment of a hair dryer 100 according to the invention. This hand dryer 100 has its air inlet 44 in a side panel 20. The nozzles are not visible in this view, but they provide an air stream in the direction of the block arrows. An LED strip 71 is provided at the rear wall 16 of the hand dryer 100 to illuminate and decontaminate the nozzles. With hand dryers 100 of this type, most of the water from the user's hands is blown onto the wall to which the hand dryer 100 is attached or on the floor below the hand dryer 100. If the user has not thoroughly washed his hands before drying them or has touched other parts of his body or nearby surfaces after washing them, there is a risk of this wall or floor being contaminated by bacterial and microbial contamination. To reduce the health risks that may arise from such contamination, the hand dryer 100 may comprise light sources that are arranged to illuminate the immediate surroundings of the hand dryer 100 too.

1. A hand dryer comprising at least one part that is susceptible to contamination, and at least one light source for emitting light in a far UVC portion of the electromagnetic spectrum, the at least one light source being arranged in such a way as to illuminate the at least one part for the decontamination thereof, when emitting the light.

2. A hand dryer as claimed in claim 1, wherein the at least one light source is configured for emitting light with a wavelength of about 222 nm.

3. The hand dryer as claimed in claim 1, wherein
the at least one part comprises a nozzle for blowing out a hand drying air stream.

4. The hand dryer as claimed in claim 1, further comprising a controller, operatively coupled to and configured for timed control of the at least one light source.

5. The hand dryer as claimed in claim 4, further comprising a decontamination button, operatively coupled to the controller, the controller being configured to activate the at least one light source in response to activation of the decontamination button.

6. The hand dryer as claimed in claim 4, further comprising at least one touch sensor for detecting a touch of the at least one part, the touch sensor being operatively coupled to the controller, the controller being configured to activate the at least one light source in response to detecting the touch of the at least one part.

7. The hand dryer as claimed in claim 6, wherein the touch sensor is configured to detect a touch of a portion of the at least one part, the controller being configured to activate the at least one light source to selectively illuminate the touched portion.

8. The hand dryer as claimed in claim 4, further comprising a contamination detector, operatively coupled to the controller, for detecting contamination on a contaminated portion of the at least one part, the controller being configured to selectively illuminate the contaminated portion.

9. The hand dryer as claimed in claim 4, wherein the hand dryer comprises a fan, operatively coupled to the controller, for generating a hand drying air stream, the controller being configured to temporarily disable the fan while emitting the light.

10. The hand dryer as claimed in claim 1, wherein the at least one part comprises an air filter or a drip tray.

11. The hand dryer as claimed in claim 1, wherein the at least one light source is arranged in such a way as to illuminate at least a portion of a wall to which the hand dryer is attached for the decontamination thereof, when emitting the light.

12. The hand dryer as claimed in claim 1, wherein the at least one light source is arranged in such a way as to illuminate at least a portion of a floor surface below the hand dryer for the decontamination thereof, when emitting the light.
